(19) United States
(12) Patent Application Publication
(10) Pub. No.: US 2008/0053253 A1
(43) Pub. Date: Mar. 6, 2008

(54) Fully ambulatory, self-contained gait monitor

(75) Inventors: Steven T. Moore, New York, NY (US); Hamish G. MacDougall, Woolloomooloo (AU); Roberta Allen, Arnold, MD (US)

Correspondence Address: LARRY J. GUFFEY, WORLD TRADE CENER - SUITE 1800, 401 EAST PRATT STREET, BALTIMORE, MD, 21202, US

(73) Assignee: INDIVIDUAL MONITORING SYSTEMS, INC, Baltimore, MD (US)

(21) Appl. No.: 11/897,722

(22) Filed: Aug. 31, 2007

Related U.S. Application Data
(60) Provisional application No. 60/842,598, filed on Sep. 6, 2006.

(51) Int. Cl. A61B 5/00

(52) U.S. Cl. 73/865.4

(57) ABSTRACT

A gait monitoring device for recording and assessing, with the use of a personal computer, the gait characteristics of one wearing the device, includes: (a) a transducer array for sensing the temporal variation in the vertical acceleration and angular velocity of the motion of the shank of a wearer, (b) an analog to digital converter for sampling the data sensed by the transducer array, (c) a microprocessor having embedded programmable memory, (d) a sampled data storage means, (e) firmware for controlling the operation of the microprocessor to sample the output of the transducer array at a prescribed time interval and to temporarily store the sampled data, (f) a USB interface that allows for the downloading of the stored data to the personal computer, and (g) software for controlling a personal computer in the analysis of the collected data.

### CROSS-REFERENCE TO RELATED APPLICATION

This application claims the benefit of U.S. Provisional Patent Application No. 60/842,598, filed Sep. 6, 2006 by the present inventors.

### BACKGROUND OF THE INVENTION

1. Field of the Invention

The present invention generally relates to an ambulatory apparatus or device and methods for monitoring, recording and assessing an individual's stride and gait characteristics.

2. Background for Development of the Present Invention

Parkinson's Disease (PD) is a common neurodegenerative disorder reflecting a progressive loss of dopaminergic and other subcortical neurons. Clinically, PD is primarily manifested as a motor disturbance, most notably resting tremor, hypometria (reduced movement size), bradykinesia (slowness of movement), rigidity, a forward stooped posture, postural instability and freezing of gait. Levodopa, the metabolic precursor to dopamine, has commonly been used to manage the motor symptoms of PD for over forty years by replacing depleted dopamine at the striatum.

Although initially effective, as the disease advances the duration of each dose shortens (the ‘wearing off’ effect), necessitating more frequent administration. In addition, the development of dyskinesias (involuntary movements) and the ‘off/on’ phenomenon (abrupt and unpredictable responses to individual doses of levodopa) can significantly affect the quality of life in PD patients and complicate dosing. Moreover, impairment of locomotor function in PD restricts movement and increases the risk of falling, producing the most significant lifestyle disturbance−loss of safe mobility.

The goal of the neurologist in managing motor dysfunction in PD is to manipulate the dopaminergic dosing schedule to minimize ‘off’ periods, without inducing dyskinesias due to excessive dopamine in the brain. One means of formulating a patient's optimal levodopa dosing schedule is to base it upon observations of the patient's gait over various periods of time. However, to date no objective method exists for making and recording such observations.

Linear accelerometers have been used for long-term monitoring of motor fluctuations in PD, in its simplest form as an activity monitor worn on the wrist or belt. More recent studies have employed multi-axis, wrist-mounted accelerometers to distinguish hypokinesia (lack of voluntary movements), bradykinesia, and tremor during patient activity in the home, although ‘on’ and ‘off’ phases could not be reliably determined in individual subjects. A more ‘brute-force’ approach to accelerometry (six tri-axial accelerometers; mounted on both upper arms, both upper legs, the sternum and one wrist) could distinguish between ‘on’ and ‘off’ stages of PD, using a neural network to identify the motor states of bradykinesia, hypokinesia and tremor at one-minute intervals. A similar approach could also distinguish dyskinesias from voluntary movements. However, gross body acceleration data does not indicate the functional locomotor capacity of the individual; i.e., how well the patient is walking.

One of the cardinal features of PD is locomotor dysfunction; shortened stride length, increased variability of stride, shuffling gait, and freezing. To characterize pathological gait in the PD patient it is necessary to accurately monitor stride length. Clinical and research studies have measured stride over short intervals; however, data obtained in a laboratory setting can provide only a ‘snapshot’ of gait characteristics, which may fluctuate markedly in PD patients over the course of a day.

A number of ambulatory systems have employed gyroscopes to measure the angular velocity of the thigh and/or shank, and integrated these waveforms to obtain the angular extent of leg swing, which when scaled by subject height yields a somewhat inaccurate estimate of stride length. Some improvements in accuracies have been achieved by utilizing gyroscopes on the shank of both legs and a third gyroscope on the thigh. However, cables used to relay data from gyroscopes to a central logging unit create an unacceptable trip hazard and interfere with patients' normal daily activity.

Commercial ambulatory systems utilizing accelerometers do exist and are capable of estimating average stride length over an epoch (e.g., the IDEEA LifeGait System of Minisun LLC, Fresno CA; the AMP331 monitor of Dynastream Innovations Inc, Alberta Canada). However, the stride length estimations from such devices have been found to lack sufficient accuracy to enable what are herein referred to as “stride-by-stride measurements”.

From a review of the prior art pertinent to the present invention, it is clear that there continues to be a need for new and improved quantitative means and methods for monitoring, recording and assessing an individual's stride and gait characteristics. This applies to all situations where stride and gait should be measured, particularly in evaluating any disorder (i.e., not just PD) that affects stride and gait.

3. Objects and Advantages

There has been summarized above, rather broadly, the background that is related to the present invention in order that the context of the present invention may be better understood and appreciated. In this regard, it is instructive to also consider the objects and advantages of the present invention.

It is an object of the present invention to provide apparatus and methods for the long-term ambulatory monitoring of pathological gait, suitable for clinical evaluation of PD.

It is also an object of the present invention to develop a fully ambulatory, self-contained monitor of gait that measures stride lengths, acceleration and velocity, speed of strides, vertical and horizontal frequencies and enables one to detect step hesitation and ‘freezing’ in PD patients.

It is a further object of the present invention to develop a fully ambulatory, self-contained monitor that evaluates gait over successive steps so as to measure gait characteristics and diagnose/identify gait abnormalities occurring both with PD and also in other conditions affecting gait.

It is an object of the present invention to provide apparatus and methods for evaluating the severity of gait abnormalities occurring both with PD and also in other conditions affecting gait.

It is also an object of the present invention to provide apparatus and methods for assessing the benefits for various treatments to reduce or eliminate gait abnormalities.

It is an additional object of the present invention to provide apparatus and methods for evaluating ‘freezing’ incidents in PD patients.

These and other objects and advantages of the present invention will become readily apparent as the invention is better understood by reference to the accompanying summary, drawings and the detailed description that follows.

### SUMMARY OF THE INVENTION

Recognizing the need for the development of improved apparatus and methods for the long-term ambulatory monitoring of an individual's gait, the present invention is generally directed to satisfying the needs set forth above and overcoming the limitations seen in the prior art gait monitoring device and methods.

In accordance with a preferred embodiment of the present invention, an improved gait monitoring device for recording and assessing, with the use of a personal computer, the gait characteristics of one wearing the device, includes: (a) a transducer array for sensing the temporal variation in the vertical acceleration and angular velocity of the motion of the foot of the wearer, (b) an analog to digital converter for sampling the data sensed by the transducer array, (c) a microprocessor having embedded programmable memory, (d) a sampled data storage means, (e) firmware for controlling the operation of the microprocessor to sample the output of the transducer array at a prescribed time interval and to temporarily store the sampled data, (f) a USB interface that allows for the downloading of the stored data to the personal computer, and (g) software for controlling the operation of the personal computer to analyze the sampled data to determine the wearer's gait characteristics.

In a further refinement of the present invention, its software is configured so as to analyze measurable gait characteristics chosen from the group consisting of: a) the length of every stride taken by the wearer over an extended period of time, b) the variability in these stride lengths over this period of time, c) the times during the period when the length of the strides are less than a defined percentage of what can be computed to be the baseline value of the stride lengths, or d) the impact on the wearer's stride by his/her consumption of a dose of medication.

Thus, there has been summarized above, rather broadly and understanding that there are other preferred embodiments which have not been summarized above, the present invention in order that the detailed description that follows may be better understood and appreciated.

### BRIEF DESCRIPTION OF THE DRAWINGS

FIG. 1 shows an embodiment of the present invention located on a patient's shank.

FIG. 2 is a schematic diagram of a preferred embodiment of the present invention.

FIG. 3 shows, at various instances, one's leg movements, which determine the person's stride length, and the vertical linear accelerations and pitch angle velocity measurements that were collected by the present invention in monitoring these leg movements.

FIG. 4 shows on the top line the vertical linear accelerations, measured with a preferred embodiment of the present invention, for the left shank of a patient with advanced PD at three different periods: quiet standing, gait initiation and FOG. Shown on the line below are the frequency spectra of these accelerations for each of these periods.

### DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

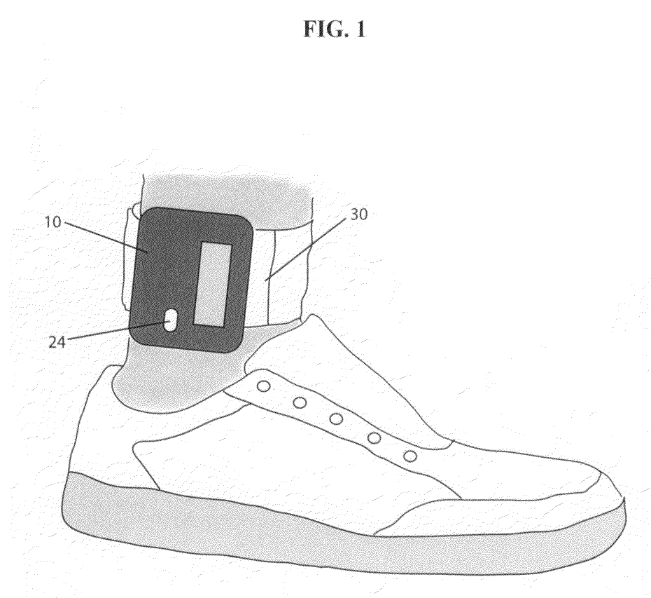

In a preferred embodiment, the present invention takes the form of a fully ambulatory, microcontroller-based, stride and gait evaluation monitor (SAGE-M) that is a small, self-contained device (weighing less than 100 grams and approximately the size of a pager) that is mountable on the shank just above the ankle. See FIG. 1. The SAGE-M acquires and stores linear acceleration and angular velocity of the measured leg at a sample rate of 100 Hz over a period of up to 24 hours. USB connectivity allows the later uploading of data to a PC and analysis software (SAGE-S) provides an accurate measure of every stride taken by the subject over the recording epoch. Data on consecutive strides can characterize Parkinsonian gait and provide a dynamic assessment of the wearer's/patient's locomotor response to therapy, allowing an objective evaluation of pharmacological, surgical, and rehabilitation interventions that could be used to adjust ongoing treatments.

The device utilizes a combined accelerometer/gyroscope sensor array that is mounted on a patient's leg. This device provides improved accuracy (5 cm) over a wide range of stride length (0.2-1.5 m) by using a three stage process: (i) vertical acceleration of the shank detects periods of locomotion; (ii) an initial stride length estimate is calculated by integration of the gyroscope angular velocity signal, and (iii) a final accurate stride length value is determined using a novel calibration algorithm that accounts for forward motion of the body over the stance foot.

Frequency analysis of shank vertical acceleration data can be used to detect episodes of freezing. Meanwhile, custom analysis software is used to processes stride data to provide clinically-relevant information on the PD patient's response to dopaminergic therapy, such as latency from administration to improved stride length, abruptness of transition from ‘off’ to ‘on’ (using an Emax function), and time spent in the ‘off’ state.

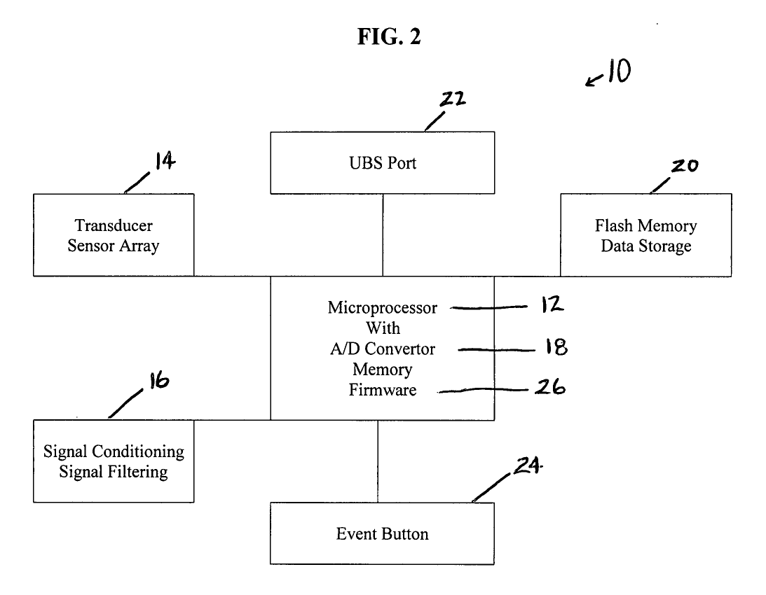

A preferred embodiment of the present invention 10 includes an 8 bit, on board programmable flash microcontroller 12, a transducer array 14 that includes a ±6 g accelerometer (a triaxial package in which only one channel is logged and which has a frequency response 0-40 Hz), a ±1200°/sec angular gyroscopic velocity sensor (frequency response 0-40 Hz), appropriate signal conditioning and filtering circuitry 16, a 12 bit A/D converter 18 which has a 100 Hz signal sampling rate, a 35 Mbyte flash memory 20 having 24 hour recording capacity at 400 bytes/second, a USB 2.0 interface 22 (alternatively, the data could be wirelessly transmitted to a remote personal computer), a AAA 9V NiMH rechargeable battery that is chargeable through the USB port, and an external event button 24 to allow a user to flag the occurrence of 1 s an event (medication administration, freezing episode, etc) that is pertinent to the analysis of the wearer's gait characteristics. See FIG. 2.

The firmware 26 that was developed for the present invention utilized structured programming implemented in C and assembly language. It is interrupt driven firmware and includes the following functions: (a) timing clock, (b) A/D conversion ready, (c) USB port data reception, (d) command setting with host computer, (e) synchronization of start of recording time with host computer (upon command), (f) during recording, sequential memory storage of the following data blocks at a constant 100 Hz rate: vertical acceleration (12 bits), angular rate (12 bits), event status (8 bits).

The present invention is also equipped with a elasticized strap 30 having a hook and loop fastener that allows the unit to be mounted around a patient's shank (e.g., just above the ankle).

Data can be transferred (e.g., using a USB cable) to a personal computer (PC) and processed using a Windows-based interface and custom analysis software (SAGE-S) written in Labview G (National Instruments, Austin, Tex.). The interface aspect of this software allows the user to program the start date and time for data acquisition, enter patient information into a data file, upload a data file to a PC using the USB connection, and check SAGE-M battery status.

The analysis aspect of the present invention enables one to use a leg's vertical acceleration and angular velocity measurements to compute any one of a host of clinical parameters relating to PD gait dysfunction and a patient's response to dopaminergic therapy.

A calibration algorithm is used to correct for movement of the body over the stance foot to determine the length of the stride. Other clinical parameters of interest include: stride length variability, ‘off’ time (when the stride length is less than 50% (a defined percentage) above a baseline value), latency of locomotor response to levodopa, abruptness of transition from ‘off’ to ‘on’, etc.

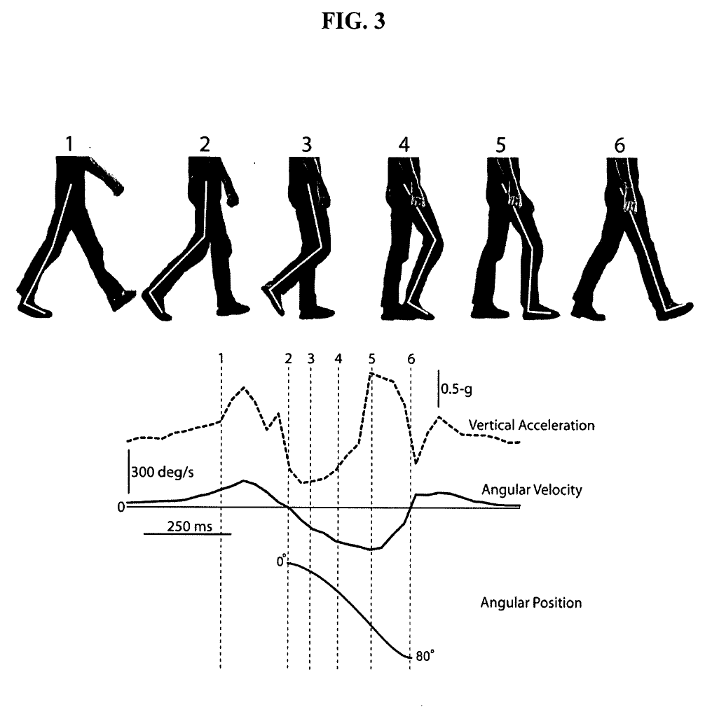

The performance of the present invention is illustrated in FIG. 3. It shows, at various instances, video images of one's leg movements and, at the same time, the data from the present invention which is measuring, for the left leg on which the monitor is attached at the shank, the leg's vertical linear acceleration (dashed line) and pitch angular velocity (solid line). These measurements are used to determine the person's stride length by equating periods of negative angular velocity to the forward rotation of the leg during its swing (during upright stance there was a DC offset of 9.8 m/s² in the vertical acceleration, and changes in this DC component were used to detect when the patient was supine). Locomotor activity is defined herein as occurring during those periods where the root-mean square (RMS) vertical acceleration of the unit is greater than 0.4 m/s².

An estimate of the angular extent of leg swing was obtained by integration of the negative portion of the angular velocity trace during periods of locomotion (as determined from the RMS vertical acceleration). An initial stride length estimate (SLi) was calculated as follows:

SLi=2×l×sin(α/2) (1)

where l is the length of the leg from the trochanter (hip joint) to the ground, which can be measured directly or estimated as 53% of participant height, and a is the angular extent of the swing phase.

Determining stride length from leg swing alone is reasonably accurate for small stride lengths (<1 m) as there is minimal forward motion of the pelvis during the swing phase. However, this technique underestimates larger strides due to the considerable forward motion of the body over the stance foot in addition to the component generated by leg swing. To overcome this problem, a novel calibration algorithm was developed that could provide accurate stride length measurements from a single, shank-mounted gyroscope. See Moore et al., “Long-term Monitoring of Gait In Parkinson's Disease,” Gait & Posture, 26, pp 200-207 (2007).

A ‘one-size-fits-all’ group calibration algorithm was developed for clinical uses of the present invention and where individual calibrations were not practical (e.g., for patients with advanced PD). This “group calibration” algorithm utilizes a direct measure of the stride length obtained from 10 healthy participants walking along a 30 meter hallway. Healthy participants/controls were utilized as it was necessary to acquire angular velocity data over a wide range of stride lengths (˜0.2-1.5 m) from each participant.

An aluminum tube was taped to the heel of the left shoe and a whiteboard marker inserted such that the tip left a single dot on the floor during each foot placement (pen technique). Simultaneous estimates of stride length were obtained from the present invention attached to the left leg. Actual stride length was determined from measurement of the distance between successive dots on the floor. Participants were instructed to walk at a natural pace but to vary gait according to verbal commands to produce a wide range of stride lengths, including small shuffling steps typical of Parkinson's disease. The pen technique was chosen as it enabled the calibration over a wide range of stride lengths, was relatively accurate (˜5 mm error), and enabled calibration outside of the laboratory.

Plotting height-normalized true-versus-estimated stride lengths from the ten controls revealed a non-linear but consistent relationship, such that it was possible to generalize a correction algorithm applicable to all participants. To correct for forward motion of the body over the stance foot, a least-squares fit (Labview Advanced Analysis Package, National Instruments, Austin Tex.) was applied to the height-normalized initial stride length estimate (SLₙᵢ) of the form:

$${SL}_{nc} = {a}_{0} + {a}_{1} sin ( {SL}_{ni}^{2} ) + {a}_{2} 3 cos ( {SL}_{ni} ) + \frac{{a}_{3}}{{SL}_{ni} + 1} + {a}_{4} {SL}_{ni}^{4} \qquad ( 2 )$$

where SL_{nc} is the height-normalized corrected stride length, and the group calibration coefficients aᵢ were [−43.34, 21.86, 14.91, −1.42, 2.25]. The mean error was 2.8% of participant height (maximum error 9%), or 5 cm for the average participant height of 167 cm.

Application of Equation (2) to the height-normalized initial stride estimates and multiplication by participant height yielded an accurate stride measure over the full range of stride length. The error per stride was also estimated by comparing the total distance traveled down the hallway (cumulative stride length of the true and corrected values) and dividing by the number of strides taken for each participant. Mean error was similar to that calculated from the height-normalized data at 4.8 cm, with a maximum error of 8 cm.

An alternative to a group calibration is to derive the coefficients of Equation (2) for each individual subject. Using the data from the ten control subjects, individual subject calibration algorithms were computed and were found to reduced the mean error by 33%, from 4.8 cm to 3.2 cm. Thus, if increased accuracy is required subjects can be individually calibrated rather than using the group calibration coefficients. However, this may not be possible for patients with advanced PD, who cannot vary stride over a sufficient range to provide adequate calibration.

The present invention (SAGE-M) was then used to obtain stride data from two PD participants in the ‘off’ state (no dopaminergic medication in the previous 12 hours). A participant with a relatively mild form of PD walked a distance of 4.5 m (5 strides) and simultaneous pen and SAGE-M measures of stride length (left leg) were obtained. The average stride length was 90.1 cm (pen) and 89.2 cm (SAGE-M). A second participant with severe locomotor impairment traversed a distance of 89 cm utilizing small shuffling steps (7 strides) that yielded an average stride length of 12.7 cm (video analysis) and 10.4 cm (SAGE-M), and the mean difference was 2.5 cm. Thus, at two extremes of locomotor impairment in the PD ‘off’ state, the accuracy of the present invention was within that established in the ten healthy controls.

Differences between healthy and Parkinsonian gait over extended periods were also monitored with the present invention. Over four hours a healthy participant covered a total of 3.9 km with 3071 strides. Stride length was stable at 1.5 m and consistent with the typical value for adult males. In contrast, four hours of data from a PD patient during normal daily activities outside of the clinic demonstrates the cardinal features of Parkinsonian gait; namely a small (˜0.5 m), highly variable stride length, covering a distance of 492 m with 923 strides.

Freezing of gait (FOG) and falls in PD patients are generally thought to be closely related; both occur sporadically, are often resistant to dopaminergic treatment, and greatly diminish quality of life. Recent studies have demonstrated a high-frequency movement of the leg (2-6 Hz) during FOG, which may be preceded by higher stride-to-stride variability. To date there is no objective measure of FOG and subsequent falls outside the laboratory.

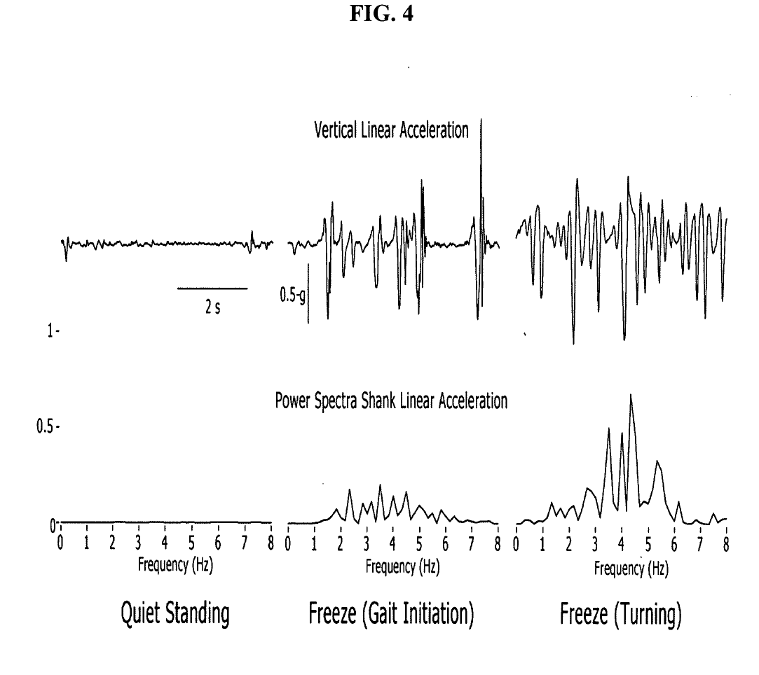

Using a preferred embodiment of the present invention, a pilot study (N=11) demonstrated that FOG could be identified in PD subjects from the appearance of high frequency components (2-6 Hz band) in the vertical acceleration of the leg that were not apparent during quiet stance or walking. See FIG. 4 which shows on the top line the vertical linear accelerations, measured with a preferred embodiment of the present invention, for the left shank of a patient with advanced PD at three different periods: quiet standing, gait initiation and FOG. Shown on the line below are the frequency spectra of these accelerations for each of these periods. The high frequency movement of the leg (2-6 Hz) during FOG are readily apparent.

Thus, the potential exists for the present invention to be used for extended real-time monitoring of gait in PD that can both identify FOG and predict an impending FOG episode, based on high-frequency vertical leg acceleration and changes in stride length, respectively. See Moore et al., “Ambulatory Monitoring of Freezing of Gait in Parkinson's Disease,” Movement Disorders, 22, Suppl. 16, pp. S78-79(2007).

The foregoing is considered as illustrative only of the principles of the invention. Further, since numerous modifications and changes will readily occur to those skilled in the art, and because of the wide extent of the teachings disclosed herein, the foregoing disclosure should not be considered to limit the invention to the exact construction and operation shown and described herein. Accordingly, all suitable modifications and equivalents of the present disclosure may be resorted to and still considered to fall within the scope of the invention as hereinafter set forth in claims to the present invention.

We claim:

1. A gait monitoring device for recording and assessing, with the use of a personal computer, the gait characteristics of one wearing said device, said device comprising:
a means for sensing the temporal variation in the vertical acceleration and angular velocity of the motion of said wearer at the location where said device is being worn,
an analog to digital converter connected to said sensing means for sampling the data sensed by said motion sensing means,
a microprocessor connected to said converter, said microprocessor having embedded programmable memory,
a means for storing said sampled data,
a firmware means for controlling the operation of said microprocessor to 1 s sample the output of said sensing means at a prescribed time interval and to temporarily store said sampled data in said data storage means, and
a means for transferring said stored data to said personal computer.

2. The gait monitoring device as recited in claim 1, wherein said device configured so as to be worn on the shank of said wearer.

3. The gait monitoring device as recited in claim 1, further comprising a software means for controlling the operation of said personal computer to analyze said sampled data to determine said gait characteristics of said wearer.

4. The gait monitoring device as recited in claim 2, further comprising a software means for controlling the operation of said personal computer to analyze said sampled data to determine said gait characteristics of said wearer.

5. The gait monitoring device as recited in claim 3, wherein said software means is configured so as to analyze gait characteristics chosen from the group consisting of: a) the length of every stride taken by a wearer over an extended period of time, b) the variability in said stride lengths over said period of time, c) the times during said period when the length of said strides are less than a defined percentage of what can be computed to be the baseline value of said stride lengths, d) the impact on said stride lengths by the consumption of a dose of medication by said wearer, or e) the frequency spectra of said vertical accelerations and the identification of episodes of “freezing gait” in said wearer's movements.

6. The gait monitoring device as recited in claim 4, wherein said software means is configured so as to analyze gait characteristics chosen from the group consisting of: a) the length of every stride taken by a wearer over an extended period of time, b) the variability in said stride lengths over said period of time, c) the times during said period when the length of said strides are less than a defined percentage of what can be computed to be the baseline value of said stride lengths, d) the impact on said stride lengths by the consumption of a dose of medication by said wearer, or e) the frequency spectra of said vertical accelerations and the identification of episodes of “freezing gait” in said wearer's movements.

7. The gait monitoring device as recited in claim 5, wherein said software means includes a calibration algorithm in said stride length analysis that accounts for the forward motion of said wearer's body over the foot making said stride.

8. The gait monitoring device as recited in claim 6, wherein said software means includes a calibration algorithm in said stride length analysis that accounts for the forward motion of said wearer's body over the foot making said stride.

9. The gait monitoring device as recited in claim 1, wherein said sensing means including a transducer array that includes an accelerometer and a gyroscopic sensor.

10. The gait monitoring device as recited in claim 8, further comprising a means for temporally indicating the occurrence, during said monitoring, of an event that is relevant to the analysis of said gait characteristics.

11. A method for recording and assessing, with the use of a personal computer, the gait characteristics of an individual of interest, said method comprising the steps of:
sensing the temporal variation in the vertical acceleration and angular velocity of the motion at a specified location on said individual,
sampling with an analog to digital converter at a prescribed frequency said sensed accelerations and angular velocities,
storing said sampled data,
controlling said sensing, sampling and data storage with a microprocessor having embedded programmable memory, and
transferring said stored data to said personal computer.

12. The method as recited in claim 11, wherein said specified location is on the shank of said individual.

13. The method as recited in claim 11, further comprising the step of controlling, with appropriate software, the operation of said personal computer to analyze said sampled data to determine said gait characteristics of said individual.

14. The method as recited in claim 12, further comprising the step of controlling, with appropriate software, the operation of said personal computer to analyze said sampled data to determine said gait characteristics of said individual.

15. The method as recited in claim 13, wherein said analysis of said gait characteristics includes characteristics chosen from the group consisting of: a) the length of every stride taken by a wearer over an extended period of time, b) the variability in said stride lengths over said period of time, c) the times during said period when the length of said strides are less than a defined percentage of what can be computed to be the baseline value of said stride lengths, d) the impact on said stride lengths by the consumption of a dose of medication by said wearer, or e) the frequency spectra of said vertical accelerations and the identification of episodes of “freezing gait” in said wearer's movements.

16. The method as recited in claim 14, wherein said analysis of said gait characteristics includes characteristics chosen from the group consisting of: a) the length of every stride taken by a wearer over an extended period of time, b) the variability in said stride lengths over said period of time, c) the times during said period when the length of said strides are less than a defined percentage of what can be computed to be the baseline value of said stride lengths, d) the impact on said stride lengths by the consumption of a dose of medication by said wearer, or e) the frequency spectra of said vertical accelerations and the identification of episodes of “freezing gait” in said wearer's movements.

17. The method as recited in claim 15, wherein said analysis includes using a calibration algorithm in said stride length analysis that accounts for the forward motion of said wearer's body over the foot making said stride.

18. The method as recited in claim 16, wherein said analysis includes using a calibration algorithm in said stride length analysis that accounts for the forward motion of said wearer's body over the foot making said stride.

19. The method as recited in claim 11, wherein said sensing step includes utilizing a transducer array that includes an accelerometer and a gyroscopic sensor.

20. The method as recited in claim 18, further comprising the step of utilizing a means for temporally indicating the occurrence, during said monitoring, of an event that is relevant to the analysis of said gait characteristics.
